(19) United States
(12) Patent Application Publication
(10) Pub. No.: US 2026/0259157 A1
(43) Pub. Date: Sep. 3, 2026

(54) METHOD AND DEVICE FOR NON-DESTRUCTIVELY TESTING A CONNECTION POINT IN A COMPONENT ASSEMBLY

(71) Applicant: EJOT SE & CO. KG, Bad Berleburg (DE)

(72) Inventors: Mario MAIWALD, Creuzburg (DE); Marco WERKMEISTER, Georgenthal / OT Schoenau v.d.W (DE); Toni MUELLER, Gotha (DE)

(21) Appl. No.: 18/835,931

(22) PCT Filed: Feb. 7, 2023
(86) PCT No.: PCT/EP2023/052894

§ 371 Date: Aug. 5, 2024

(30) Foreign Application Priority Data
Feb. 8, 2022 (DE) 10 2022 102 940.9

(51) Int. Cl. G01N 25/18; B23K 20/12; B23K 31/12; G01J 5/00; G01J 5/48; G01J 5/80; G01N 25/72; G01N 33/207; G06T 7/00

(52) U.S. Cl. CPC G01N 25/18; B23K 31/125; G01J 5/48; G01J 5/80; G01N 25/72; G06T 7/001; B23K 20/1295; G01J 2005/0077; G01N 33/207; G06T 2207/10048; G06T 2207/30152

(57) ABSTRACT

In a method for non-destructively testing a connection point in a component assembly, the connection point includes a base layer and at least one cover layer, and a connection element with a head and a shaft, the connection point being formed between the shaft of the connection element and the base layer. The cover layer has a thermal conductivity greater than the base layer and the connection element. The test is carried out via a thermal imaging camera and an inductor arranged on the component assembly side opposite the camera. The inductor excites the base layer at an excitation time using a pulse via an active surface. The method incorporates: at a first detection time, capturing a reference image of the connection point via the thermal imaging camera, the head being imaged as a head surface area; at a second detection time, capturing a test image analogous to the reference image, the second detection time occurring after the first detection time and a specified duration after the excitation time; defining an analysis region in the test image and/or in the reference image, the analysis region at least partly comprising the head surface, and the analysis region being divided into sub-regions, in particular pixels, the sub-regions being assigned a temperature value; forming a temperature change value using a rule based on the temperature values of the corresponding sub-regions of the reference and test image; based on the temperature change value, forming an “OK” value if the temperature change lies in a pre-defined temperature interval with at least one lower boundary; classifying the connection point in that the sum of the sub-regions with an “OK” value is compared with a reference interval with at least one predetermined lower interval boundary, the connection point being classified as an “OK” connection if the sum lies in the reference interval.

This application is a national stage application filed under 35 U.S.C 371 of International Application No. PCT Application No. PCT/EP2023/052894 filed Feb. 7, 2023, which claims priority to German Patent Application No. 10 2022 102 940.9 filed Feb. 8, 2022. The disclosures of the above-referenced applications are incorporated herein by reference in their entireties.

The invention relates to a method, of the type specified in the preamble of claim 1, for non-destructively testing a connection point in a component assembly, and to a device of the type specified in the preamble of claim 23.

Non-destructive testing methods for substance-to-substance bonded connection points are already known in the art. Such methods are preferably based on the principle of thermography. The component assembly is heated by an excitation source, and the different temperature behavior of the components and the connection point allows conclusions to be drawn about the connection point. The temperature behavior is preferably recorded using a thermal imaging camera. An evaluation system connected to both the excitation source and the thermal imaging camera is used to evaluate and classify the connection points.

DE 10 2006 057 802 A1 discloses a method and a testing system for the non-destructive testing of resistance pressure welding connection. The substance-to-substance resistance pressure welding connection is subjected to pulsed excitation using a flash lamp. The evaluation system comprises a thermographic camera that records the temperature behavior of the substance-to-substance bond and analyses the time course of the received light intensity based on the temperature behavior.

For a better local resolution, the camera area in which the substance-to-substance bonded connection point is located is subdivided into several camera pixels by analyzing the history of each camera pixel. Evaluation and classification are performed based on time-dependent detector units. Substance-to-substance bonded connection points exhibiting insufficient connection strength, with each camera pixel featuring detector units above a certain threshold value being assigned an “OK” value, are detected in that there is a too small total area of the “OK” values in relation to another threshold value. The area is determined at a time 200 ms after excitation.

This test method can be used to determine the size or the thermally conductive connection of a pressure welding point that connects two stacked metal sheets, by recording at a specific point in time the total area with a sufficient weld. The absolute temperature is measured at a specific point in time at which the heat radiation has reached the surface of the cover layer via the welding spot.

Other methods for the non-destructive testing of substance-to-substance bonded connections by evaluation of the intensity curves over time are known in the art. For the sake of completeness only, reference is made to DE 10 2007 042 341 A1 and DE 10 2007 050 005 A1.

The prior art testing methods are mainly used for spot-welded connections or bonded connections. The connection points are substance-to-substance bonded and essentially homogeneous.

Moreover, prior art testing methods are preferably intended for precise, time-consuming, fine classification, with each testing system being designed for use in certain operating conditions only.

Non-destructive testing methods for a welded connection, in particular spot welded connections, of two component layers, which are in particular made of steel, are thus known in the art.

What is desirable now is to have a non-destructive way of reliably assessing the quality of mixed component connections. In the case of mixed component connections, two component layers made of different materials are usually connected by means of a connection element. Unlike conventional spot-welded connections, it is not the quality and size of the contact point between the two components that is important in these connections, but also the transition between the two components and the connection element.

For example, poor contact between the head of the connection element and the upper component layer—the cover layer—results in inadequate transmission of the contact pressure as well as in insufficient tightness of the connection. Such a defect can be detected by purely visual inspection.

Moreover, it is crucial for the quality of such a mixed component connection that there is sufficient strength between the connection element and the lower layer of the construction, the base layer. This property cannot be evaluated purely optically, because the connection point is located within the component assembly.

It is the object of the invention to provide a method, of the type specified in the preamble of claim 1, for non-destructively testing such a connection point in a component assembly, which method can be used to evaluate the quality of the connection with regard to the above-mentioned aspects.

It is another object of the invention to enhance a respective device of the type specified in the preamble of claim 23 in such a way that fast, easy and flexible testing of connection points within a component assembly made with a connection element is ensured, whilst avoiding the above mentioned shortcomings, and to provide a corresponding device.

This object is accomplished for a method for non-destructively testing a connection point in a component assembly by the characterizing features of claim 1, and for a device by the characterizing features of claim 23 in conjunction with the features of the respective preamble.

The dependent claims relate to advantageous further embodiments of the invention.

The temperature behavior of the component assembly changes due to the different geometric characteristics of the component assembly. Furthermore, inhomogeneities, in particular air gaps between the base layer and the cover layer, and between the connection element and the cover layer, and defects, for example cracks, result in varying degrees of thermal conduction depending on time and on location in the component assembly.

In a manner known per se, the component assembly to be tested comprises a base layer and at least one cover layer, and a connection element with a head and a shaft. In the connection point, there is a connection between the shaft of the connection element and the base layer. The connection element is positively connected to the cover layer and its head presses the cover layer against the base layer.

The connection element has a lower thermal conductivity than the cover layer. Preferably, the base layer can also have a lower thermal conductivity. This is particularly true for steel-aluminum connections, which are used in many areas of lightweight construction.

The invention is based on the realization that the quality of the connection can be assessed on the basis of the “thermal conductivity” of the component connection, namely the thermal conduction from the base layer via the connection point of the connection element with the base layer and the cover layer and the connection of the head to the cover layer.

According to the invention, testing a component connection of the type described above is carried out by means of a thermal imaging camera and an inductor which is arranged on the component assembly side opposite the thermal imaging camera. The inductor inductively excites the base layer via an active surface at an excitation time by means of an excitation pulse.

At a first detection time, a reference image of the connection point is captured by the thermal imaging camera, in which the head is shown as a head surface. At a second detection time, a test image is captured in the same way as the reference image.

The second detection time is after the first detection time and a predefined period of time after the excitation time. In particular, the second detection time represents the time at which the heat input at the head surface of an “OK” connection, which is via the cover layer, can be measured at the head surface of the connection element. In particular, the heat input via the cover layer can still exceed the heat input via the shaft of the connection element at this point in time.

An evaluation region is defined in the test image and/or in the reference image. The evaluation region at least partly comprises the head surface. The evaluation region is divided into sub-regions, in particular pixels. A temperature value each is assigned to the sub-regions. Based on the temperature values of the corresponding sub-regions of the reference and test images, a temperature change value is formed on the basis of a rule.

On the basis of the temperature change value, an “OK” value is formed if the temperature change is within a pre-defined temperature interval.

If there is sufficient heat conduction via the cover layer in the component assembly, in an “OK” connection, a sufficiently large number of sub-regions will already be found in the predefined temperature interval with at least one predetermined lower interval boundary at the second detection time, due to the higher thermal conductivity of the cover layer. There has not yet been heat equalization within the connection element, particularly in the region of the head. This suggests that there are no significant defects within the entire transmission path. This makes it possible to distinguish between an “OK” connection and a faulty connection.

In a welded connection, for example, quality defects appear in the form of so-called cracks in the transition from the base layer to the shaft, which cracks include areas of trapped air all around. As a result, heat conduction into the cover layer is reduced. In particular, there is also a change in heat generation in the base layer due to the induction in the presence of the cracks extending into the base layer, which influences the heat conduction through the component assembly.

A lack of connection of the head to the cover layer also results in reduced heat conduction via the cover layer, which is then reflected accordingly in the temperature change image at the second detection time.

Based on this, the connection point is classified in that the sum of the sub-regions with an “OK” value is compared with a reference interval. The connection point is classified as an “OK” connection if the sum is within the reference interval, otherwise the connection point is classified as a “not OK” connection. The reference interval has a lower interval boundary and is matched to the head size.

This takes account of the fact that in particular the heating of the edge area of the head reflects the heat conduction via the cover layer.

The method according to the invention thus allows the quality of a component connection to be evaluated in an easy way with just a single evaluation, taking into account both the connection point of the connection element to the base layer and the transition of the head of the connection element to the cover layer.

Preferably, the rule is designed as a subtraction of the temperature values of the corresponding sub-regions of the test image from those of the reference image; in particular, the temperature value of the sub-region of the reference image is subtracted from the temperature value of the sub-region of the test image. Subtraction makes it easy to obtain a temperature change value for each pixel.

Using the temperature change value takes account of the fact that the head has an inhomogeneous radiation behavior due to its geometry, the influence of which can be reduced by detecting the temperature change.

Preferably, the second detection time is selected such that temperature differences within the head surface will still be present at the second detection time for a “not OK” connection.

This point in time is determined in particular by evaluating the temperature curve of a connection known as an “OK” connection using a calibration measurement. This ensures that a reliable and reproducible classification of the connection point is possible.

In another advantageous embodiment of the invention, for determining the second detection time, the temperature change curve of the calibration measurement of an “OK” connection is generated from the excitation time of a sub-region, or the mean value of at least two sub-regions of the evaluation region is generated at least until the gradient of the temperature change curve becomes negative. This means that a point in time can be selected as the second detection time at which temperature equalization and saturation effects, which occur after the temperature change maximum, are reduced. For determining the second detection time, only times up to the maximum temperature change are thus taken into account, in particular the time when the maximum temperature is reached or shortly before the maximum temperature is reached.

This ensures a significant difference between the temperature change values of “OK” pixels and the remaining pixels during the test procedure, which enables a meaningful classification.

In another preferred embodiment of the invention, the rule for evaluating the temperature changes can comprise the generation of a temperature change image which shows the temperature change at the respective pixel. The generation of a temperature change image has the advantage that it allows further graphical processing, in particular image processing using filter algorithms.

Preferably, the second detection time corresponds to the time of the maximum temperature change of the temperature change curve, with the maximum temperature change being in particular at least 2 Kelvin (K), preferably more than 5 K. Setting the second detection time at the time of the maximum temperature change allows a meaningful evaluation of the individual pixels.

The temperature interval is preferably determined on the basis of the temperature change curve of the calibration measurement. This allows the connection point to be tested in a way that is adapted to the properties of the component assembly.

For example, the “OK” value can be assigned to a corresponding pixel if the temperature change of a pixel is greater than or equal to 5 to 10 Kelvin. This enables clear demarcation from surrounding regions and defects.

Preferably, the temperature curve is generated by the calibration measurement under ambient conditions prevailing when the test image is taken. This minimizes the influence of the ambient conditions during the test procedure.

Preferably, the active surface of the inductor is arranged centrally to the head surface in the base layer. This enables uniform and targeted excitation below the base layer of the connection element.

Preferably, the active surface and the thermal imaging camera are disposed in alignment. This ensures that the connection point is completely within the capture range of the thermal imaging camera.

To enable easy and automated positioning of the active surface, the inductor is positioned based on head surface contour detection by the thermal imaging camera.

The component assembly, and in particular the connection element, will heat up differently due to the ambient temperature, the different materials and geometric shapes. This different temperature distribution of the component assembly enables passive detection of the head by the thermal imaging camera, because the temperature of the head of the connection element is significantly different from the surrounding component assembly.

In another embodiment of the invention, the reference interval has an upper interval boundary. Having a second interval limit results in a more targeted evaluation of the individual pixels of the test image. This allows a distinction to be made between different types of error, or as to whether the sum of the “OK” values is below one interval boundary or above the other interval boundary. If the head of a fastener penetrates too far into the cover layer, this can lead to a bead forming around the head in the cover layer. This bead changes the structure of the cover layer to such an extent that increased radiation can be detected. This can then also indicate a quality defect.

Preferably, the at least one interval boundary is formed by a tolerance of 10% of the sum of the “OK” values of the reference measurement. This eliminates minor, irrelevant deviations between the test image and the reference measurement.

Preferably, the evaluation range corresponds to ±20% of the head surface. This means that, in particular, the edge and transition areas of the connection element are also taken into account during the test.

In another advantageous embodiment of the invention, the evaluation range is determined using a calculation based on the reference measurement. This allows the evaluation region to be defined quickly and automatically.

Preferably, the evaluation region is centered with respect to the head surface. Defining a central evaluation region with respect to the head surface will ensure at least partial evaluation of the head surface and of the cylindrical region below the head surface. This allows inhomogeneities within the region to be detected.

Preferably, the distance between the inductor and the lower layer of the component assembly is set such that a significant temperature input is generated in the component assembly. If the distance between the inductor and the component assembly is too large, no clear classification will be possible because the difference in the temperature change values is too small.

To filter out external errors in a temperature change image, a filter such as a Gaussian filter can be applied to the temperature change image. This allows the information content of adjacent pixels to be included in the evaluation.

Preferably, the component assembly is designed as a mixed construction connection, in which the base layer and the joining element made of steel form a friction-welded connection and the cover layer is made of aluminum.

In yet another advantageous embodiment of the invention, the base layer has a thickness of between 0.8 mm and 2 mm, and the cover layer has a thickness of between 1.0 mm and 4 mm.

Preferably, the connection element is designed as a friction element provided with drive structures on its head.

Another aspect of the invention relates to a device comprising a thermal imaging camera, a positioning means, an inductor and an evaluation control unit. Preferably, the excitation frequency of the inductor is between 15 kHz and 50 kHz. This prevents the excitation from penetrating too deeply into the component assembly, in particular into the connection element in the “depth range” of the cover layer. This ensures that the influence of the heat flow via the cover layer can be sufficiently taken into account.

Preferably, the evaluation control unit carries out the method according to the invention.

Preferably, the evaluation control unit controls the positioning means.

In yet another advantageous embodiment of the invention, the positioning means comprises a means of connection, in particular by means of a C bracket, of the thermal imaging camera and the inductor, which are designed to be stationary relative to each other, and the connection means is positioned relative to the connection point. This connection means enables them to be positioned accurately and automatically relative to one another.

Preferably, the positioning means comprises two independently movable sub-means, with one sub-means being connected to the thermal imaging camera, and the other sub-means being connected to the inductor and positioning the thermal imaging camera and the inductor relative to the connection point. The sub-means are preferably designed as independently movable robot arms. Thanks to this design, even difficult-to-access joints can be reached by the testing device, depending on the design of the robot arms.

Preferably, the thermal imaging camera is used to detect the exact position of the connection point, with the detected position being used to control the positioning means.

In yet another embodiment of the invention, the evaluation control unit comprises a data storage unit which, in particular, stores reference measurements and temperature profiles.

Additional advantages, features and possible applications of the present invention will be apparent from the description which follows, in which reference is made to the embodiments illustrated in the drawings. In the drawings,

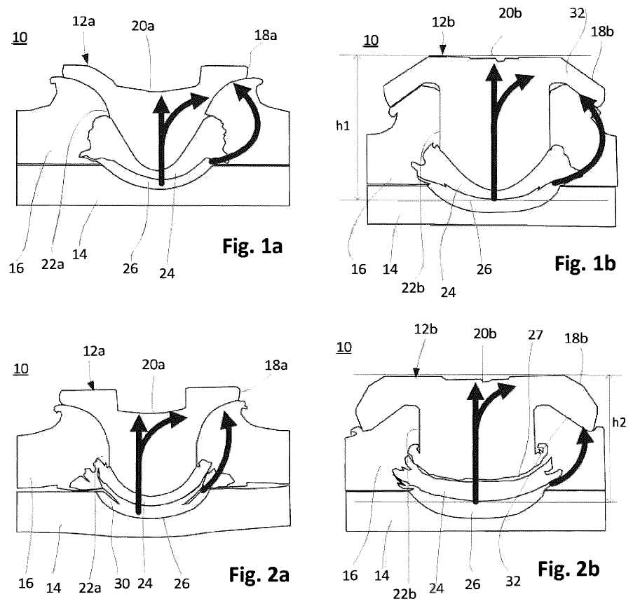

FIG. 1a is a sectional view of the component assembly with a connection element producing an “OK” connection;

FIG. 1b is a sectional view of the component assembly with another connection element producing an “OK” connection;

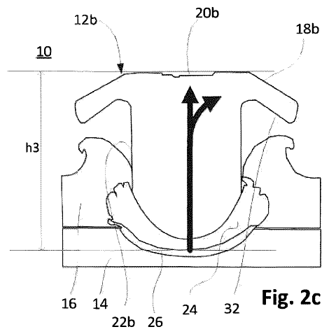

FIG. 2a is a sectional view of the component assembly with a connection element of FIG. 1a producing a “not OK” connection, in which the connection point has cracks;

FIG. 2b is a sectional view of the component assembly with a connection element of FIG. 1b producing a “not OK” connection, in which the connection point has folds;

FIG. 2c is a sectional view of the component assembly with a connection element of FIG. 1b producing a “not OK” connection, in which the penetration depth of the connection element is insufficient;

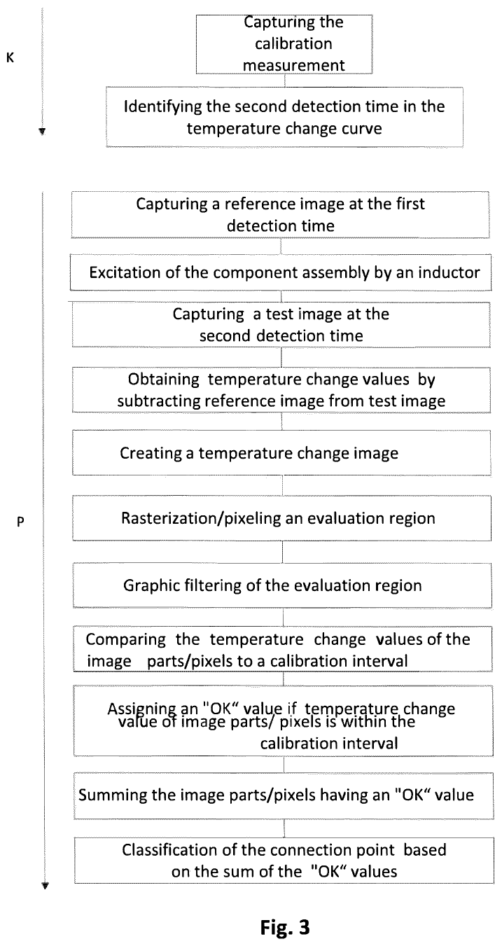

FIG. 3 is a schematic diagram illustrating the sequence of steps of the method according to the invention;

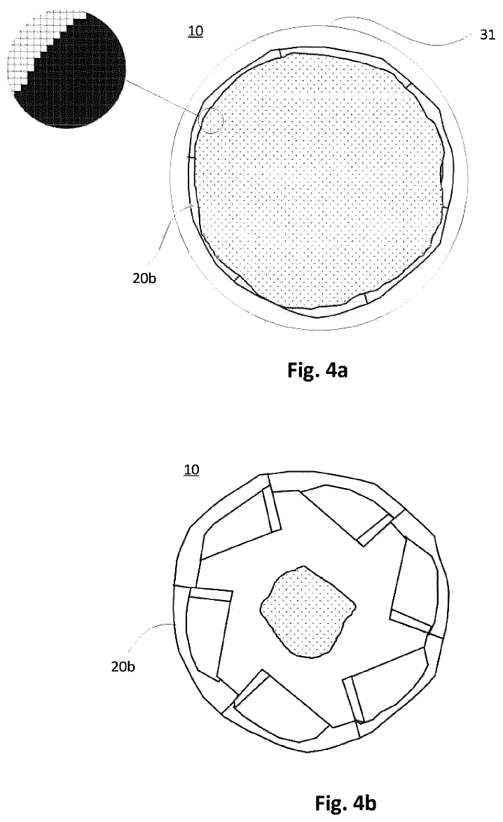

FIG. 4a is a temperature change image according to FIG. 2a with visualized “OK” values;

FIG. 4b is a temperature change image according to FIG. 2c with visualized “OK” values;

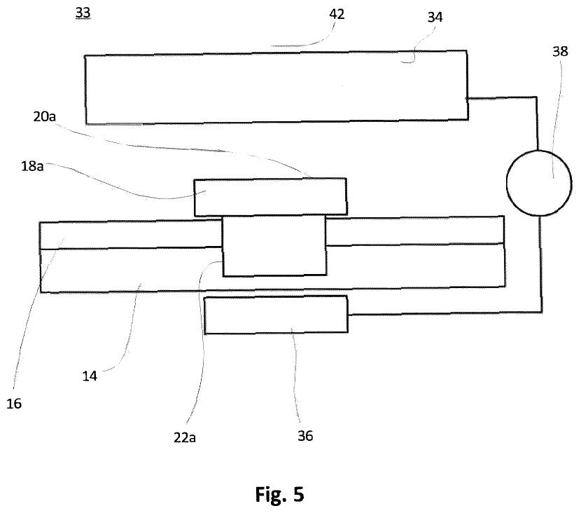

FIG. 5 is a sectional view of the device according to the invention,

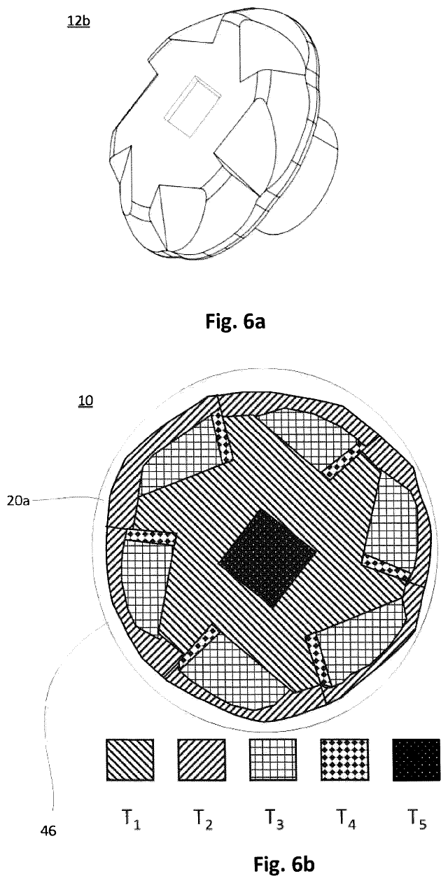

FIG. 6a is a perspective view of a second embodiment of the connection element 12b;

FIG. 6b is a test image of a reference measurement of the second embodiment of the connection element 12b of FIG. 6a with an evaluation region; and

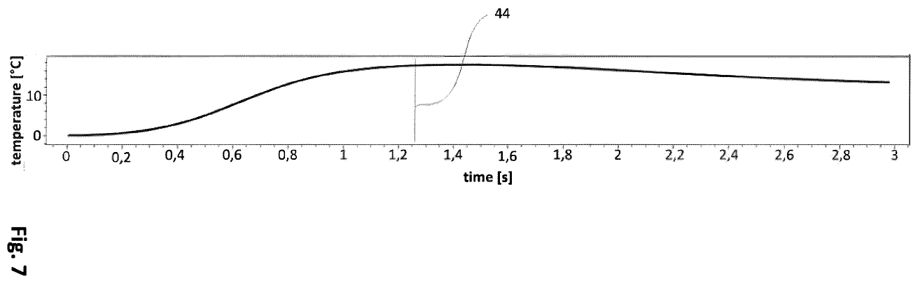

FIG. 7 is a graph of the temperature change in the evaluation region of the test image at a second detection time.

FIGS. 1a to 2c each show a schematic sectional view of a connection point 10, comprising a connection element 12a, 12b for connecting a base layer 14 to a cover layer 16, a heat-affected zone 24 of the connection element 12a, 12b and a heat-affected zone 26 of the base layer 14. The base layer 14 and the connection element 12a, 12b are formed in the same way and in particular have a similar conductivity. The cover layer 16 is arranged on the base layer 14. The connection element 12a, 12b comprises a head 18a, 18b with a head surface 20a, 20b and a shaft 22a, 22b. A heat-affected zone 24 of the connection element 12a, 12b is located in the lower area of the connection element 12a, 12b. Below the heat-affected zone 24 of the connection element 12a, 12b is a heat-affected zone 26 of the base layer 14. The connection element 12a, 12b is inserted through the cover layer 16 into the component assembly consisting of the cover layer 16 and the base layer 14 and then forms a friction-welded connection with the base layer 14.

The cover layer 16 is made of aluminum, the base layer 14 and the connection element 12a, 12b are made of steel. As a result, the component assembly is of the mixed construction type.

In one embodiment of the invention, the cover layer has a thickness of between 1.0 mm and 2 mm, and the base layer has a thickness of between 0.8 mm and 4 mm. The connection element is preferably a friction element.

The arrows in FIGS. 1a-2c each indicate heat conduction paths in the connection point 10 after the excitation time.

FIG. 1a is a view of an “OK” connection with a first embodiment of a connection element 12a, which first embodiment of the connection element 12a has a drive structure 28 for an internal drive in the head 18a of the first embodiment of the connection element 12a, and the first embodiment of the connection element 12a is designed in the manner of a countersunk head screw. The shaft 22a of the first embodiment of the connection element 12a is cylindrical. The heat-affected zone 24 of the first embodiment of the connection element 12a and the heat-affected zone 26 of the base layer 14 are homogeneous. The head 18a of the first embodiment of the connection element 12a rests on the cover layer 16 in some areas.

FIG. 1b is a view of an “OK” connection with a second embodiment of a connection element 12b, which connection element 12b differs from the first embodiment of the connection element 12a by the geometric design of the head 18b of the second embodiment of the connection element 12b and of the shaft 22b of the second embodiment of the connection element 12b. The second embodiment of the connection element 12b has a different structure in the head 18b of the second embodiment of the connection element 12b, which is provided for an external drive. The head surface 20b of the second embodiment of the connection element 12b is larger in diameter than the head surface 20a of the first embodiment of the connection element 12a. Furthermore, the head 18b of the second embodiment of the connection element 12b has a circumferential head projection 32, which is angled in the direction of the component assembly. The shaft 22b of the second embodiment of the connection element 12b is of cylindrical shape.

Heat conduction in the connection point 10, subsequent to the excitation time, essentially takes place via two paths. Firstly, via the shaft 22a of the first embodiment of the connection element 12a, with the heat having previously been conducted through the base layer 14 and the heat-affected zones 24, 26. On the other hand, the heat is conducted from the heat-affected zone 26 of the base layer 14 via the cover layer 16 into the head 18a of the first embodiment of the connection element 12a. As seen in FIGS. 1a and 1b, the heat conduction via the cover layer 18 is faster than via the shaft 22a, 22b of the connection element 12a, 12b, because the cover layer 18, in particular made of aluminum, has a higher thermal conductivity than the connection element 12a, 12b. The second detection time is selected so that a significant amount of the heat conduction of the induced heat via both paths, in particular via the cover layer, has reached the head surface.

FIG. 2a is a view of a “not OK” connection with a first embodiment of the connection element 12a. In the region of the heat-affected zone 24 of the connection element 12a and the heat-affected zone 26 of the base layer 14, the connection point 10 has cracks 30 in the lateral areas, which are formed as circumferentially flat tears, i.e. they are conical in shape.

The cracks 30 in the lateral area of the heat-affected zone 26 of the base layer 14 cause flat air pockets to form in some areas. During excitation, these air pockets lead to an inhomogeneous, ring-shaped induction in the base layer outside the cracks. This heat is mainly conducted laterally, away from the connection point 10, in the base layer 14. The cracks 30 additionally inhibit heat conduction from the heat-affected zone 26 of the base layer 14 via the cover layer 16 into the head 18a of the first embodiment of the connection element 12a. Because the cover layer 16 has a significantly higher specific heat capacity than the base layer 14, it is assumed that the heat energy in the base layer is no longer sufficient to significantly heat the cover layer 16 by the second detection time. This heat build-up prevents effective heat conduction into the cover layer 16. The heat is primarily conducted via the welded connection of the base layer 14 to the shaft 22a of the first connection element 12a, which leads to subsequent heating of the head 18a of the first embodiment of the connection element 12a. As a result, the head 18a of the connection element 12a is less heated at the second detection time than in an “OK” connection, but the heated region in the center of the head, which has undergone a temperature change required for its assessment as an “OK” pixel, is smaller, since the edge region of the head was not sufficiently heated by either the material of the cover layer or the shaft. This makes it clear that the assessment made at the second detection time is essential for the validity of the test.

This type of heat conduction also applies analogously to the second embodiment of the connection element 12b, and to any other embodiment of a connection element.

FIG. 2b shows a “not OK” connection with a second embodiment of the connection element 12b. The “not OK” connection is characterized by the fact that the second embodiment of the connection element 12b is inserted deeper into the component assembly compared to the “OK” connection of FIG. 2a (h1>h2), where h1 is the penetration depth of the second embodiment of the connection element 12b into the component assembly of FIG. 1b, and h2 is the penetration depth of the second embodiment of the connection element 12b into the component composite of FIG. 2b. As a result, the lower part of the shaft 22b of the second embodiment of the connection element 12b forms so-called folds. An air gap 27 is formed between the second embodiment of the connection element 12b and the heat-affected zone 24 of the second embodiment of the connection element 12b. Accordingly, heat conduction takes place around the air gap.

Heat conduction in the connection point 10 of FIG. 2b takes place primarily via the heat-affected zones 24, 26 through the cover layer 16 into the head 18b of the second embodiment of the connection element 12b. Heat conduction via the shaft 22b of the second embodiment of the connection element 12b is inhibited because of the air gap 27 that was formed by the deeper penetration of the connection element 12b. The deep penetration of the second embodiment of the connection element 12b additionally causes the formation of a bead from the cover layer 16 at the radially outer end of the head 18b of the second embodiment of the connection element 12b. The bead formed results in an expansion of the heated area at the second detection time. The form of heat conduction for a corresponding “not OK” connection applies equivalently to a connection point 10 with a first embodiment of the connection element 12a, or to another embodiment of a connection element.

FIG. 2c shows a “not OK” connection made with a second connection element 12b, which second connection element 12b has not been inserted as deeply into the component assembly as the “OK” connection of FIG. 2a (h1<h3), where h3 is the penetration depth of the second connection element 12b into the component assembly of FIG. 2c. Here, in comparison to FIGS. 1b and 2b, the head projection 32 is not connected to be flush with the cover layer 16.

Heat conduction in the connection point 10 of FIG. 2c essentially takes place from the heat-affected zone 26 of the base layer 14 through the heat-affected zone 24 of the first embodiment of the connection element 12a and then into the shaft 22a of the first embodiment of the connection element 12a. The heat is conducted from the shaft 22a of the first embodiment of the connection element 12a into the head 18a of the first embodiment of the connection element 12a. Firstly, due to the short penetration depth of the first embodiment of the connection element 12a, the distance (h3) over which the heat is conducted is longer than the distance (h1) of an “OK” connection of the first embodiment of the connection element 12a, and secondly, the head is not engaged with the cover layer 16 in a heat-transferring manner.

This means that, although there is an intact welded connection over the entire shaft surface, the head 22a of the first embodiment of the connection element 12a will nevertheless only be heated fully and sufficiently at a later point in time, after the second detection time, owing to the poor contact between the head and the cover layer.

In this way, a single measurement is sufficient to rule out the presence of both superficial and internal quality defects.

The “not OK” connection of FIG. 2a with the first embodiment of the connection element 12a is also classified as a “not OK” connection with the second embodiment of the connection element 12b by the test method according to the invention. The defects of the connection points 10 of FIGS. 2a to 2c can also be detected by the test method according to the invention in other embodiments of connection elements and classified as “OK” and “not OK” connections.

FIG. 3 is a schematic diagram illustrating the steps of the method for non-destructive testing. This method for testing connection points 10 is based on the principle of passive thermography, in which the component assembly is nevertheless thermally excited on the side of the base layer 14 facing away from the cover layer 16 by an excitation pulse from an inductor 36 at an excitation time which includes the region of the shaft 22a, 22b of the connection element 12a, 12b.

The second detection time 44 is determined by a calibration measurement K. The calibration measurement K is carried out using a sample known to be an “OK” connection. The connection element 12a, 12b, the thicknesses of the base and cover layers 14, 16 and the material of the base and cover layers 14, 16 are identical to the ones of the component assembly as tested in the corresponding test method P during the calibration measurement K. A calibration evaluation region 46, which includes at least part of the head surface 20a, 20b, is defined for the calibration measurement K. The calibration measurement K can be carried out at any time before the test procedure. Furthermore, the calibration measurement K can also be carried out several times, for example after a set period of time or a set number of test cycles.

The temperature change within the calibration evaluation range 46 is recorded over time. The generated temperature change curve, see FIG. 4b, contains a time of the maximum temperature change. This time is used as the second detection time 44 for the non-destructive testing procedure.

At a first detection time, which is preferably before the excitation time, a reference image of the connection point 10 with pixels to which temperature values are assigned is captured by the thermal imaging camera 34.

In particular, the first detection time is immediately before the excitation time. The thermal imaging camera 34 is arranged on the side of the cover layer 16 facing away from the base layer 14, above the head of the connection element.

The excitation causes heat conduction through the base layer 14 and then along different transfer paths/heat conduction paths to the head 18a, 18b of the connection element 12a, 12b. On the one hand, the heat is conducted into the shaft 22a, 22b of the connection element 12a, 12b via the two heat-affected zones 24, 26. The heat then flows to the head 18a, 18b of the connection element 12a, 12b. On the other hand, the heat is conducted from the heat-affected zone 26 of the base layer 14 via the cover layer 16 into the head 18a, 18b of the connection element 12a, 12b. Owing to the different materials of the cover layer and the connection element 12a, 12b, the two heat conduction paths have different thermal conduction times. The thermal conduction times also differ in particular by the material arrangement and defects, see FIG. 2a to 2c. Owing to the higher thermal conductivity coefficient of aluminum (~160 W/mK) compared to steel (~40 W/mK), heat is conducted faster in aluminum. Furthermore, there are differences in thermal conduction time due to different insertion depths of the connection elements 12a, 12b in the component assembly.

At a predetermined second detection time 44, a test image analogous to the reference image of the junction 10 is captured by the thermal imaging camera 34.

After capturing the test image, the temperature values of the reference image are subtracted from the corresponding temperature values of the test image. This can also be done automatically by the detection device when the test image is captured. Corresponding temperature change values are now assigned to the pixels of the test image, and a temperature change image, which is evaluated in an evaluation region 31, is created. Owing to the different thermal conduction times of the heat conductors and heat output losses within the component assembly for “OK” connections, see FIGS. 1a and 1b, and “not OK” connections, see FIG. 2a to 2c, the test images show significantly different temperature change distributions in each respective case.

A filter is applied to the evaluation region 31 to reduce external errors, such as chips lying on the head. The evaluation region 31 includes at least part of the head surface 20a, 20b of the connection element 12a, 12b and is centrally located in relation to the head surface 20a, 20b of the connection element 12a, 12b. The filtered pixels within the evaluation region 31 are evaluated using a temperature interval.

The temperature interval and the reference interval are based on the evaluation of a reference measurement of a test arrangement known to be an “OK” connection.

Pixels with a temperature change value that is within the temperature interval are assigned an “OK” value. In this case, pixels with a temperature change value of greater than or equal to 5.4° C. and less than or equal to 10.0° C. are assigned an “OK” value. In this range, a significant statement can be made about the heating behavior. The sum of the “OK” values in an evaluation region 31 is then compared with a reference interval. If the sum of the “OK” values is within the reference interval, the connection is “OK”. If the sum of the “OK” values is outside the reference interval, the connection point 10 is classified as a “not OK” connection. The reference interval at which a connection is assessed to be an “OK” connection is selected so that the number of pixels corresponds to between 90% and 110% of the number of pixels of the head surface.

In this case, the evaluation region 31 is designed as +20% of the head surface.

FIG. 4a is a temperature change image of an “OK” connection according to FIG. 2a with visualized “OK” values. The “OK” values are located within the head surface 20b of the second connection element 12b. The rasterized evaluation region 31 with visualized “OK” values is also shown in an enlarged detail. The evaluation region 31 includes the head surface 20b of the second connection element 12b.

FIG. 4b is a temperature change image of a “not OK” connection according to FIG. 2c with visualized “OK” values. The “OK” values are located within the head surface 20b of the second connection element 12b. Compared to FIG. 4a, the sum or region of the “OK” values is smaller and is no longer within the reference interval. This classifies the connection point as a “not OK” connection. The evaluation region 31 is rasterized analogous to the enlarged detail of FIG. 4a, and the “OK” values are visualized accordingly.

FIG. 5 is a schematic sectional view of the device 33 according to the invention with a component assembly comprising a connection point 10, for example with a connection element 12b. The device 33 comprises an inductor 36, a thermal imaging camera 34, an evaluation control unit 38 and a positioning means (not shown).

The evaluation control unit has a data storage unit which stores the results of the reference measurements and calibration measurements for the respective component assembly. Preferably, these measurements are carried out for a large number of different configurations of the connection point 10 and stored in the data storage unit. For example, the configurations differ in the connecting means 12a, 12b and/or the thickness of the base layer 14 and/or the cover layer 16.

The thermal imaging camera 34 and the inductor 36 are each electrically connected to the evaluation control unit 38. The positioning means (not shown), which has a robot arm, for example, is also electrically connected to the evaluation control unit 38.

The inductor 36 is arranged on the side of the base layer 14 facing away from the cover layer 16. The distance of the inductor from the base layer 14, the diameter and the power of the inductor 36 are set in such a way that a significant temperature input is generated within the shaft diameter, the active surface.

The thermal imaging camera 34 is arranged on the side of the cover layer 16 facing away from the base layer 14. The position and distance of the thermal imaging camera 34 relative to the component assembly is selected so that the connection point 10, in particular the head surface 20a, 20b of the connection element 12a, 12b, is located in the recording area of the thermal imaging camera 34. The thermal imaging camera 34 can also be used to detect the position of the connection point 10, in particular the head surface 20a, 20b of the connection element 12a, 12b.

The effective surface of the inductor 36 is aligned with the thermal imaging camera 34 along axis 42. The positioning means is used to position the inductor 36 and the thermal imaging camera 34 relative to each other and relative to the connection point 10. The thermal imaging camera 34 and the inductor 36 are connected in a fixed position by means of the positioning means.

In yet another embodiment, the position of the inductor 36 and the position of the thermal imaging camera 34 can each be controlled independently of one another by a sub-means of the positioning means.

FIG. 6a is a view of the second embodiment of the connection element 12b, showing in particular the indentations in the region of the head.

FIG. 6b is a schematic image of the head surface 20b of the second connection element 12b, which was recorded during the test measurement. A calibration evaluation region 46 is shown in the image. The calibration evaluation region 46 comprises the head surface 20b of the second embodiment of the connection element 12b. FIG. 6b shows the different temperatures on the head surface 20b of the second embodiment of the connection element 12b at the second detection time. Here, the following applies: T1<T2<T3<T4<T5.

T5 and T4 represent the temperature peaks, as these are depressions in the component surface, see FIG. 6a. The temperature differences result from the basic head geometry and other geometric structures, such as indentations.

FIG. 7 is a diagram of the temperature change curve of the average temperature change within the calibration evaluation region 46. The x-axis represents the time in s, and the y-axis represents the temperature change in ° C. The temperature change curve is shown for 3 s, with the maximum temperature change being approx. 18° C. The reference image was taken at time 0 s. The section line shown in the diagram represents the second detection time 44 for the test procedure. This is set between the inflection point and the maximum temperature change of the temperature change curve.

The excitation comprises an excitation period with an induction frequency of between 15 kHz and 60 kHz. The excitation duration is preferably between 0.1 s and 0.95 s. The excitation amplitude depends on the material thickness and the desired temperature change.

The method for non-destructive testing and the corresponding device 33 ensure fast, easy and flexible testing of connection points 10 in a component assembly with a connection element 12a, 12b, which allows both internal and external quality defects to be ruled out.

1. A method for non-destructively testing a connection point in a component assembly, the connection point comprising a base layer and at least one cover layer, and a connection element with a head and a shaft, the connection point being formed between the shaft of the connection element and the base layer, wherein the cover layer has a thermal conductivity which is greater than that of the base layer and of the connection element, wherein the test is carried out by means of a thermal imaging camera and an inductor which is arranged on a component assembly side opposite the thermal imaging camera, wherein the inductor inductively excites the base layer at an excitation time by means of a pulse via an active surface, the method comprising the steps of:
at a first detection time, capturing a reference image of the connection point via the thermal imaging camera, the head being imaged in said reference image as a head surface;
at a second detection time, capturing a test image analogous to the reference image, said second detection time occurring after the first detection time and a specified duration after the excitation time;
defining an analysis region in at least one of the test image and in the reference image, wherein the analysis region at least partly comprises the head surface, and the analysis region is divided into sub-regions of pixels, said sub-regions being assigned a temperature value;
forming a temperature change value using a rule on the basis of the temperature values of the corresponding sub-regions of the reference and test images;
on the basis of the temperature change value, forming an “OK” value is formed if the temperature change lies in a pre-defined temperature interval with at least one lower boundary;
classifying the connection point in that the sum of the sub-regions with an value is compared with a reference interval with at least one specified lower interval boundary, wherein the connection point is classified as an connection if the sum lies in the reference interval, otherwise the connection point is classified as a connection.

2. A method of claim 1, wherein the rule is defined as a subtraction of the temperature values of the corresponding parts of the test image from those of the reference image, in particular the temperature value of the part of the reference image is subtracted from the temperature value of the part of the test image.

3. A method as claimed in claim 1, wherein the predefined time period is selected such that, at the second detection time, there are still temperature differences within the head surface in a “not OK” connection, by evaluating the temperature curve of an “OK” connection.

4. A method as claimed in claim 1, wherein, in order to determine the second detection time, the temperature change curve of an connection from the time of excitation of a sub-region, or the mean value of at least two sub-regions of the evaluation region, is generated at least until the slope of the temperature change curve becomes negative.

5. A method as claimed in claim 4, wherein the second detection time corresponds to the time of the maximum temperature change of the temperature change curve, the maximum temperature change being greater than or equal to 2 K.

6. A method as claimed in claim 4, wherein the temperature interval is determined on the basis of the temperature change curve.

7. A method as claimed in claim 1, wherein the “OK” value is assigned at a temperature change of greater than or equal to 5 to 10 Kelvin of a pixel.

8. A method as claimed in claim 1, wherein the temperature curve is generated under the ambient conditions prevailing when the test image is taken.

9. A method as claimed in claim 8, wherein the active surface is disposed centrally relative to the head surface.

10. A method as claimed in claim 1, wherein the active surface is disposed in alignment with the thermal imaging camera.

11. A method as claimed in claim 10, wherein the active surface is positioned based on contour detection of the head surface by the thermal imaging camera.

12. A method as claimed claim 1, characterized in that the reference interval has an upper interval boundary.

13. A method as claimed in claim 12, wherein the at least one interval boundary is formed by a tolerance of 10% of the sum of the “OK” values of the reference measurement.

14. A method as claimed in claim 1, wherein the evaluation region equals ±20% of the head surface.

15. A method as claimed in claim 14, wherein the evaluation region was determined by means of a calculation based on the reference measurement.

16. A method as claimed in claim 1, wherein the evaluation region is disposed centrally relative to the head surface.

17. A method as claimed in claim 1, wherein the distance of the inductor from the component assembly is set so as to generate a significant temperature input in the component assembly.

18. A method as claimed in claim 1, wherein a temperature change image is generated and a filter is applied to the temperature change image to filter out external errors.

19. A method as claimed in claim 1, wherein the component assembly is in the form of a mixed construction joint connection.

20. A method as claimed in claim 19, wherein the base layer is made of steel and the cover layer is made of aluminum.

21. A method as claimed in claim 1, wherein the base layer has a thickness of between 0.8 mm and 2 mm, and the cover layer has a thickness of between 1.0 mm and 4 mm.

22. A method as claimed claim 1, wherein the connection element is designed as a friction element.

23. A device comprising a thermal imaging camera, a positioning means, an inductor and an evaluation control unit, wherein the evaluation control unit performs a method for non-destructively testing a connection point in a component assembly, the connection point comprising a base layer and at least one cover layer, and a connection element with a head and a shaft, the connection point being formed between the shaft of the connection element and the base layer, wherein the cover layer has a thermal conductivity which is greater than that of the base layer and of the connection element, wherein the inductor which is arranged on a component assembly side opposite the thermal imaging camera, the inductor inductively excites the base layer at an excitation time by means of a pulse via an active surface, the method comprising the steps of:
at a first detection time, capturing a reference image of the connection point via the thermal imaging camera, the head being imaged in said reference image as a head surface;
at a second detection time, capturing a test image analogous to the reference image, said second detection time occurring after the first detection time and a specified duration after the excitation time;
defining an analysis region in at least one of the test image and in the reference image, wherein the analysis region at least partly comprises the head surface, and the analysis region is divided into sub-regions of pixels, said sub-regions being assigned a temperature value;
forming a temperature change value using a rule on the basis of the temperature values of the corresponding sub-regions of the reference and test images;
on the basis of the temperature change value, forming an “OK” value if the temperature change lies in a pre-defined temperature interval with at least one lower boundary;
classifying the connection point in that the sum of the sub-regions with an “OK” value is compared with a reference interval with at least one specified lower interval boundary, wherein the connection point is classified as an “OK” connection if the sum lies in the reference interval, otherwise the connection point is classified as a “not OK” connection.

24. A device as claimed in claim 23, wherein the evaluation control unit controls the positioning means.

25. A device as claimed in claim 24, wherein the positioning means comprises a C bracket, of the thermal imaging camera and the inductor, which are designed to be stationary relative to one another, and the C bracket is positioned relative to the connection point.

26. A device as claimed in claim 23, wherein the positioning means comprises two independently adjustable sub-means, one sub-means being connected to the thermal imaging camera and the other sub-means being connected to the inductor and positioning the thermal imaging camera and the inductor relative to the connection point.

27. A device as claimed in claim 23, wherein the thermal imaging camera is designed to detect the position of the connection point, the detected position being used to control the positioning means.

28. A device as claimed in claim 23, wherein the evaluation control unit comprises a data storage unit which in particular stores reference measurements and temperature curves.
